(19) United States
(12) Patent Application Publication
(10) Pub. No.: US 2008/0286831 A1
(43) Pub. Date: Nov. 20, 2008

(54) SAMPLE COLLECTOR AND TESTER

(76) Inventor: Greg Liang, Rancho Cucamonga, CA (US)

Correspondence Address: GRANT ANDERSON LLP;C/O PORTFOLIOIP, P.O. BOX 52050, MINNEAPOLIS, MN, 55402, US

(21) Appl. No.: 12/014,577

(22) Filed: Jan. 15, 2008

Related U.S. Application Data
(60) Provisional application No. 60/885,006, filed on Jan. 15, 2007.

(51) Int. Cl. C12Q 1/04; C12M 1/34

(52) U.S. Cl. 435/34; 435/288.7

(57) ABSTRACT

Provided are devices and methods for testing solid, semi-solid, or liquid animal or environmental specimens. The multi-part device comprises a breakable seal that separates a specimen treatment chamber from a testing chamber.

### PRIORITY AND INCORPORATION BY REFERENCE

Priority is claimed to U.S. Provisional Application No. 60/885,006, filed Jan. 15, 2007, by Greg Liang, which is hereby incorporated by reference herein in its entirety.

U.S. Provisional Application No. 60/761,580, by Greg Liang and Yahong Liang, having a postcard receipt stamped with the date Jan. 23, 3006, entitled Rapid Test Apparatus, and related U.S. patent application Ser. No. 11/625,813 are also hereby incorporated by reference herein in their entirety.

### FIELD OF THE INVENTION

The invention is in general directed to devices and methods for testing solid, semi-solid, or liquid animal or environmental specimens.

### BACKGROUND

The testing of solid, semi-solid, or liquid specimens such as stool, blood, urine, saliva, or swab specimens of the cervix, urethra, nostril, or throat, as well as environmental specimens, such as food products, soil and dust, often requires pre-treating the specimens with a treatment buffer. Pre-treating the specimens modifies the specimen physically or chemically, helps to dilute the specimen, extracts substances to be detected from the specimen, reduces interfering substances, preserves the specimen, or changes the test substance into a more readily detectable form. This pre-treatment results in a new sample solution that is suitable for the test substance to be detected. Typically, the collected specimen is pre-mixed with the treatment buffer in a container separate from the test device used to detect the presence of a particular test substance. In most testing protocols, a portion of the resulting sample solution is transferred to a second test location for reacting with a reagent to obtain a test result that indicates the presence or quantity of the test substance in the specimen. For example, in a fecal occult blood test, a plastic tube is used to suspend the fecal specimen in a treatment buffer, which dissolves the blood components of the specimen. A tip end of the plastic container is then severed and a portion of the sample solution is released from the tube to a second device, which is used to conduct an immunological hemoglobin test.

The steps for severing the specimen treatment container and transferring the sample solution from the container complicate the test methods by requiring multiple steps, which expose the operator and the test area to the risk of fecal specimen contamination. These methods are not convenient for onsite testing by non-laboratory trained users. Also, transferring the sample solution may lead to inaccurate results because of the possible transfer of inaccurate test volumes. What is needed is a simpler, safer, and more accurate method of testing of solid, semi-solid, or liquid specimens.

### SUMMARY

The present invention provides devices and methods for treating and testing specimens that are simpler, safer to use, and more accurate than traditional specimen testing devices and methods. The methods are suitable for onsite and offsite sample collection, followed by testing either onsite or offsite. By providing for a two-part device, for example, a specimen treatment module may be used to collect the specimen by the patient. The module may then be provided by the patient to a laboratory, or other medical office employee, a more trained individual, for example, who then inserts the specimen treatment module in to the test module to perform the test and interpret the test result. In this manner, the patient may provide the sample in a sanitary container, yet does not need to perform the test. This allows for counseling by the patient's physician, nurse, or other medical provider. In other methods, both components may be provided to the patient, and the patient may conduct the test offsite.

In one embodiment, the device comprises a specimen treatment module for treating the specimen with a treatment buffer forming a sample solution of the specimen, and a test module comprising an absorbent reagent member in a container capable of receiving the specimen treatment module and facilitating testing of the sample solution. A treatment buffer comprises a liquid reagent or a mixture of liquid-liquid or a liquid-solid mixture comprising reagents selected from the group consisting of water, salts, pH buffers, surfactants, oxidizing or reducing agents, enzymes, enzyme substrates, chelating agents, and preservatives. The treatment buffer may be supplied in the specimen treatment module, or it may be supplied separately and added to the specimen treatment module before or after the specimen is collected in the specimen treatment chamber. In certain embodiments, one or more solutions may be mixed in the specimen treatment chamber to form the treatment buffer. Such multi-part treatment buffers are known to those of ordinary skill in the art. For example, in a throat swab streptococcus test, two buffer solutions, a sodium nitrite solution and an acetic acid solution are mixed immediately before the throat specimen is treated by the mixture solution. When the treatment of a specimen requires a mixture of more than one buffer solution, one buffer solution may be pre-stored in the specimen treatment chamber and the other buffer solution may be added at the time the specimen is being treated. For example, a second buffer solution may be added before the specimen is collected in the specimen treatment chamber.

The test module container may be any shape capable of receiving the specimen treatment module, and may, for example, be in the form of a cup, or, for example, a tube. The specimen treatment module comprises an upper open end and a lower end closed with a breakable seal, and a specimen treatment chamber defined by the sidewall, the upper open end and the breakable seal. The specimen treatment module may further comprise a cover means, such as, for example, a cap, for retaining the sample or a treatment buffer inside the specimen treatment chamber. The specimen treatment module may further comprise a specimen sampling stick for obtaining solid or semi-solid samples. The specimen treatment module may be of any suitable material, including, for example, plastic, such as, for example, a plastic selected from the group consisting of polyethylene, polypropylene, polystyrene, polyvinyl, and acrylonitrile butadiene styrene. The specimen treatment module may be any shape capable of being received by the test module container, and may, for example, be in the form of a cup, or, for example, a tube. The absorbent reagent member comprises an absorbent matrix comprising a wick section and a test section comprising a test area having at least one test reagent in the test area capable of reacting with the test substance or test substance residual of the treated specimen. The test module container comprises an upper open end and a lower closed end. The absorbent reagent member is attached to the interior side of the sidewall of the container with the wick section oriented toward the lower closed end and the test section oriented toward the upper open end of the container. The test module is capable of receiving the specimen treatment module by inserting a section of the specimen treatment module proximal to the lower end of the specimen treatment module into the test module from its upper open end. The device further comprises a seal breaking means capable of breaking the breakable seal of the specimen treatment module to allow the sample solution flow from the specimen treatment module through the wick section to the test section of the absorbent reagent member.

When treating a specimen with the device of the invention, the specimen is introduced into the specimen treatment module and mixed with the treatment buffer to form a sample solution, which can be immediately tested or stored inside the specimen treatment module for future testing. The sample solution of the specimen treatment module is tested by inserting the lower section of the specimen treatment module into the test module and breaking the breakable seal of the specimen treatment module with the seal breaking means. In the test position of the device, the assembly of the modules is positioned substantially upright. The sample solution flows, for example, by gravity, from the specimen treatment module through the broken seal of the specimen treatment module to the wick section of the absorbent reagent member, which is capable of wicking through the wick section to the test section of the absorbent reagent member.

The specimen treatment module may further comprise a filter within the chamber defined by the sidewalls, the upper end, and the closed lower end. The filter may be used to filter liquid samples, such as, for example, urine or saliva before the sample enters the test module of the device.

In the test position of the device, the test module provides a contained space for the sample solution flow and the test reaction; therefore, leakage of the sample solution to the exterior of the device is controlled. Only part of the wick section of the absorbent reagent member submerges in the sample solution. The volume of the sample solution that reacts with the test reagent and the flow rate are self-controlled by the wick properties of the absorbent reagent member.

The breakable seal of the specimen treatment module is a hydrophobic barrier made of any appropriate material, for example, the material may be selected from a the group consisting of plastic, rubber, and foil, attached to the sidewall of the specimen treatment module, which caps, or plugs the specimen treatment module. The seal breaking means for breaking the breakable seal includes any means capable of penetrating, tearing, or removing the entire breakable seal or a portion of the breakable seal. In an embodiment of the invention, the seal breaking means is a stick inserted from the upper end of the specimen treatment module capable of breaking the seal. The stick, in exemplary embodiments, is designed to be sanitary, that is, to not contaminate the specimen or sample solution. In another embodiment of the invention, the seal breaking means is a structure attached to the test module container capable of breaking the seal when the specimen treatment module is inserted into the test module.

The absorbent reagent member comprises a wick section and a test section made of at least one absorptive material. The test section comprises at least one reagent capable of reacting with the test substance to produce a test result of the presence or quantity of the test substance in the specimen. The absorptive reagent member may be, for example, selected from a group comprising lateral flow test strips, chemical and enzymatic test strips, which are known to those in the field. By “detecting” the presence of a substance is meant that the reagent member detects the presence of either a substance or an analyte that indicates the presence of the substance.

In another embodiment of the invention is provided a self-contained specimen test device for conveniently treating and testing a specimen. The components of this embodiment, such as the breakable seal and the cups or tubes, may be made of any suitable materials, including those described herein for other embodiments of the invention. The device comprises an upper end and a lower end, a test chamber containing an absorbent reagent member, a specimen treatment chamber containing a treatment buffer, with the test chamber and the specimen treatment chamber in a substantial side-by-side arrangement and separated at the bottom end of the specimen treatment chamber by a breakable seal, a cover means for retaining the treatment buffer inside the specimen treatment chamber, and a seal breaking means for breaking the breakable seal. By removing the cover means to the specimen treatment chamber, a specimen is introduced into the specimen treatment chamber and mixed with the treatment buffer to form a sample solution containing the treated specimen. The absorbent reagent member comprises an absorbent matrix comprising a wick section and a test section, at least one test reagent in the test section capable of reacting with the test substance of the specimen. With the device in a test position, the upper ends of the test chamber and the specimen treatment chamber are oriented upward; breaking the breakable seal will result in the sample solution flowing from the specimen treatment chamber to the lower end of the test chamber and contacting the wick section of the absorbent reagent member. The sample solution wicks up the absorbent reagent strip to the test area and reacts with the test reagent. As a result, an assay signal of the presence or quantity of the test substance of the specimen is produced at the test section.

The breakable seal is a breakable barrier that separates the specimen treatment chamber and the test chamber at the lower end of the specimen treatment chamber and prevents the treatment buffer from entering the test chamber before the device is used for testing a specimen.

The seal breaking means is a structure, part, or a moving mechanism that with the device in a test position is capable of breaking the breakable seal. By “breaking” in the embodiments of the present invention, is meant, for example, but not limited to, penetrating, tearing, altering, or removing part or all of the breakable seal so that the treatment buffer of the specimen treatment chamber is capable of flowing to the test chamber and contacting the wick section of the absorbent reagent member.

In one embodiment of the invention, the seal breaking means is a stick disposed in the specimen treatment chamber capable of being moved and breaking the breakable seal.

In another embodiment of the invention, the seal breaking means is a structure disposed in the test chamber capable of breaking the breakable seal.

The devices of the invention are suitable for testing specimens selected, for example, from the group consisting of stool, plasma or serum, blood, spinal fluid, urine, saliva, microbe culture media, and swab specimens of surfaces of an animal, such as the cervix, urethra, nostril, and throat, as well as environmental specimens, such as food products, soil and dust samples.

By animal is meant, for example, any live or dead animal including, for example, a mammal, for example, a human. Substances to be tested in these specimens include but are not limited to fecal occult blood components, hapto-hemoglobin complex, antibodies, bacteria, such as, for example, but not limited to, H. pylori, viruses, enzymes, proteins, hormones, tumor markers, cardiac markers, drugs, substances of abuse, allergens, pesticides, and pollutants.

Thus, in one embodiment of the invention is provided a specimen test device comprising a specimen treatment module comprising a tubular part having a sidewall, an upper open end, and a lower end closed with a breakable seal; and a test module comprising a container comprising an upper open end, a lower end, a sidewall, and a reagent member attached to the interior side of the sidewall; wherein the specimen treatment module may be inserted into the upper open end of the test module. The specimen test device may further comprise a treatment buffer, which, for example, may be supplied in a separate container or vial. The treatment buffer may, for example, be pre-stored in the specimen treatment module. The test device may further comprise a cap. In another aspect of the invention, the test device may further comprise a specimen sampling stick. In exemplary aspects of the invention, the specimen sampling stick is attached to the cap, in other exemplary aspects, the specimen sampling stick is movably attached to the cap, such that, for example, the stick may be pushed further into the specimen treatment module. In such an embodiment, the stick may, for example, be used to break the breakable seal.

In certain embodiments of the invention, the lower end of the test module further comprises an upward edge for breaking the breakable seal of the specimen treatment module.

The specimen tested using the specimen test device may be, for example, solid, semi-solid, or liquid. The device may further comprise a filter attached to the sidewalls of the specimen treatment module, wherein said filter may filter a liquid sample added to the specimen treatment module.

More than one reagent member may be used in the test device. Thus, the specimen test device may comprise at least 1, 2, 3, 4, 5, 6, 7, 8, 9, or 10 reagent members. For example, the device may comprise at least 2, or at least 3-5 reagent members. The reagent member may, for example, comprise a wick section and a test section comprising an assay reagent. The reagent member may, for example, detect the presence of an analyte that indicates the presence of a substance selected from the group consisting of fecal occult blood components, hapto-hemoglobin complex, antibodies, bacteria, viruses, enzymes, proteins, drugs, substances of abuse, allergens, pesticides, and pollutants. In certain exemplary embodiments, the reagent member detects the presence of fecal occult blood.

In other aspects of the invention, the specimen treatment module further comprises a sealing structure on the exterior of the tubular part, wherein when the specimen treatment module is inserted into the test module, the sealing structure fits to the sidewalls of the test module. In other embodiments, the sampling stick further comprises a plunger structure, wherein said plunger fits within the interior of the sidewalls of the specimen treatment module.

Also provided in the present invention is a method for testing an analyte in a specimen, comprising treating the specimen with a treatment buffer in the specimen treatment module of a device of the present invention, inserting the specimen treatment module into the test module; breaking the breakable seal of the specimen treatment module; allowing the specimen and specimen treatment buffer to flow through the seal into the test module; and detecting the presence of the analyte by reading the results on the reagent member. In some aspects, the method further comprises closing the specimen treatment module with a cap and shaking the specimen treatment module. In some aspects, the method further comprises opening the cap to allow the specimen and specimen treatment buffer to flow through the seal. In exemplary embodiments, the specimen is fecal matter and, for example, the reagent member detects analytes in fecal occult blood.

Also provided in the present invention is a kit comprising the specimen treatment module of the present invention. The kit may further comprise treatment buffer and a cap. In certain aspects, the kit may further comprise a cap and a specimen sampling stick. The kit may, for example, further comprise treatment buffer. The kit may further comprise instructions. In certain embodiments, the kit further comprises packaging for providing the device for testing. The kit may contain more than one specimen treatment modules. For example, the kit may comprise at least 2, 3, 4, 5, 6, 7, 8, 9, or 10 specimen treatment modules. Or, for example, the kit may comprise 2-3, 2-4, 2-5, 2-6, 2-7, or 2-8 specimen treatment modules. In other aspects of the invention the kit may comprise the device of the invention, thus the kit comprises one or more specimen treatment modules and one or more test modules of the present invention. The kit may, for example, comprise from 1-10 specimen treatment modules and from 1-10 test modules.

### BRIEF DESCRIPTION OF THE DRAWINGS

FIG. 1 depicts an elevated perspective view of a specimen treatment module of a device of the invention.

FIG. 2 depicts an elevated perspective view of a test module of a device of the invention.

FIG. 3 depicts an elevated perspective view of a device of the invention with the specimen treatment module and the receiver module in a test position.

FIG. 4 is an elevated view of an embodiment of a device of the invention and elevated views of the parts of the device.

FIG. 5 is an elevated view of a different embodiment of a device of the invention.

### DETAILED DESCRIPTION

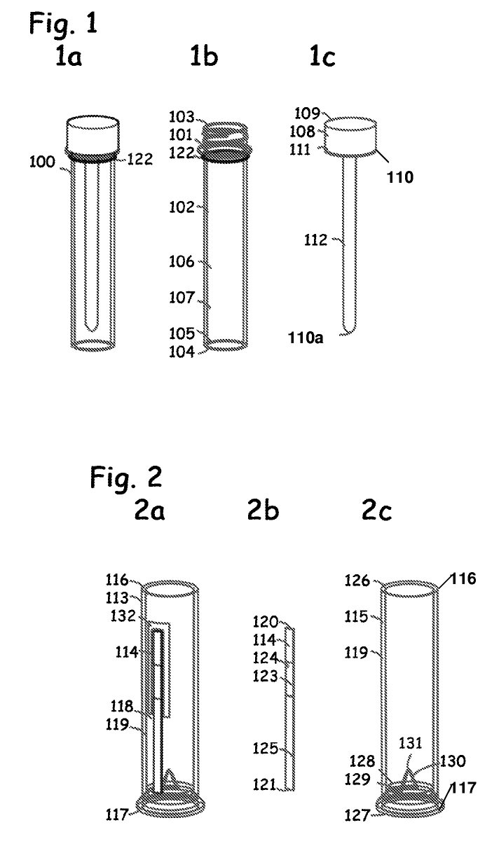

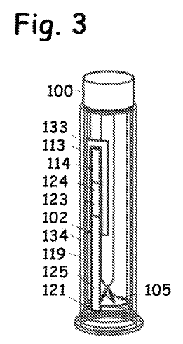

FIG. 1, in conjunction with FIG. 2 and FIG. 3, depicts an embodiment of a device of the invention comprising a specimen treatment module and a test module.

FIG. 1 consists of drawings of the specimen treatment module 100 (1a) of the device, which comprises a tubular part 101 (1b) having a sidewall 102, an upper open end 103 and a lower end 104 closed with a breakable seal 105, a specimen treatment chamber 106 defined by the sidewall 102, the upper open end 103, and the breakable seal 105 containing a treatment buffer 107, and a cover means 108 (1c). The cover means 108 comprises an upper end 109 and lower end 110, a plug section 111 proximal to the lower end 110 sized to fit the upper open end of the tubular part 103 and prevent the treatment buffer 107 from flowing out of the chamber 106, and a sampling stick section 112 for conveniently collecting a specimen by contacting the specimen with its lower end 110a. The collected specimen is transferred to the interior of the tubular part 101 by inserting the sampling stick into the interior of the tubular part from the upper open end 103 and closing the upper open end with the plug section 111. The mixture of the specimen and the treatment buffer forms a sample solution for testing. A rubber o-ring 122 as a sealing member sized to tightly fit the exterior of the middle area of the specimen treatment module is employed for filling the gap between the specimen treatment module and the test module.

FIG. 2 consists of drawings of a test module 113 (2a) comprising an absorbent reagent member 114 (2b), and a container 115 (2c) comprising an upper open end 116 and a lower end 117 with the reagent member 114 attached to the interior side 118 of the sidewall 119. The reagent member 114 comprises an upper end 120 and a lower end 121, a test section 123 proximal to the upper end 120 comprising a reagent 124 and a wick section 125 proximal to the lower end 121. The container of the test module 115 comprises an upper open end 126 and a lower end 127. Bottom 128 comprises a reagent member fixture 129 and a seal breaking structure 130 comprising an upward edge 131 for breaking the breakable seal 105 of the specimen treatment module 101 when the device is in a test position (FIG. 3). The upward edge comprises a surface sharp enough to break the seal 105. The reagent member 114 is held to the interior 118 of the sidewall 119 of the container 115 by the reagent member fixture 129 and a tape 132. It is understood that means other than tape, such as, for example, an adhesive or a plastic slot structure, may be used to hold the reagent member to the interior of the sidewall.

FIG. 3 depicts the device 133 of the invention in a testing position. The specimen treatment module 100 is inserted into the test module 113. The reagent member 114 resides in the chamber 134 between the sidewalls 102 and 119 of the specimen treatment module and container of the test module. The breakable seal 105 is torn by the seal breaking structure 130. The sample solution is capable of flowing from the specimen treatment module through the broken seal 105 to the lower end 121 of the reagent member and wicking to the test section 123. The test result of the test substance of the specimen is readable at the test section 123. In certain embodiments, the sample solution flows more freely through the broken seal to the test module where the air-tight cap is loosened or removed from the specimen treatment module tube.

It is understood that the present invention is not restricted to the seal-breaking means depicted in the Figures. In other embodiments, for example, the test module 113 does not comprise a seal breaking structure 130 with an upward edge 131. For example, in other embodiments, a stick may be inserted through the upper open end 103 of the specimen treatment module, and pushed downward to break the seal 105. In such embodiments, the stick may, for example, be the sampling stick 112 which may be movably connected to the cover means 108, so that it may be pushed downward to break the seal 105. Or, for example, in other embodiments, the cover means 108 does not comprise a sampling stick 112. In such embodiments, a separate sampling stick may be used, or liquid samples may be transferred using a pipette or dropper device.

In yet other embodiments of the invention, the sample stick further comprises an air-tight plunger structure, for example, an o-ring gasket that snugly fits to the interior of a section of the specimen treatment module, positioned a distance apart from the upper end of the cap on the sample stick. Upon insertion into the specimen treatment module, the plunger structure will slide down the inside of the upper section of the tube and pressurizes the interior of the tube. As a result, the air between the treatment buffer in the specimen treatment module and the O-ring is compressed. When the specimen treatment module is inserted into the test module, and the bottom seal is broken, the expanding air propels the sample solution from the specimen treatment module into the test module. The distance between the upper end of the cap and the position of the plunger structure is appropriate to properly pressurize the air within the tube and expels sufficient volume of sample solution from the specimen treatment chamber into the test chamber. One of ordinary skill in the art may determine the proper placement based on the dimensions of the modules of the device.

Optionally, the specimen treatment module comprises a septum within the interior of the specimen treatment chamber that separates the specimen treatment chamber into two sections, a lower section containing the treatment buffer and an upper section without treatment buffer. A hole in the middle of the septum about the same diameter of the sample stick allows the specimen stick to pass through and retains excess specimen from entering the treatment buffer. One of ordinary skill in the art would be able to determine how to construct such a septum (Zhou et al, U.S. Pat. No. 6,921,370).

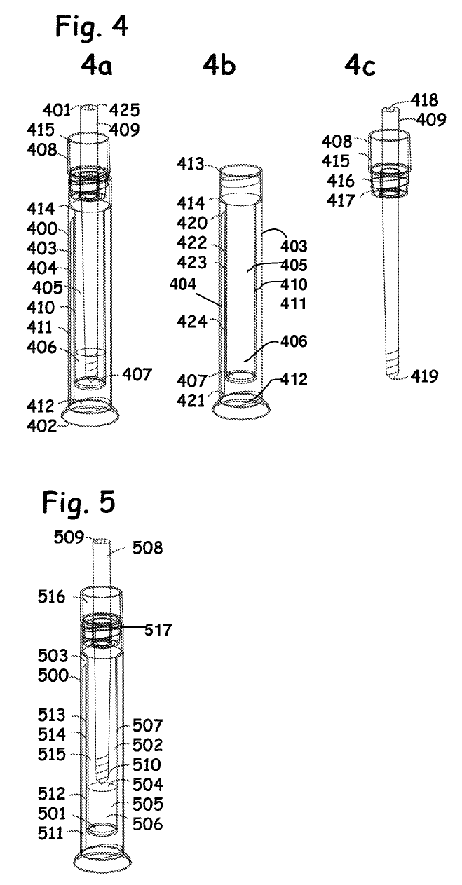

FIGS. 4 and 5 depict embodiments of a self-contained device of the invention.

FIG. 4 depicts an embodiment of a specimen test device 400 (FIG. 4a) of the invention. The specimen test device 400 comprises an upper end 401 and a lower end 402, an assembly (FIG. 4b) comprising a test chamber 403, an absorbent reagent member 404, and a specimen treatment chamber 405, a breakable seal 407 separates the test chamber and the specimen treatment chamber, and an assembly (FIG. 4c) comprising a cover means 408 and a seal breaking means 409. The test chamber 403 contains the absorbent reagent member 404. The specimen treatment chamber 405 comprises a treatment buffer 406. The test chamber 403 is defined by the interior sidewall 410, an exterior sidewall 411, the breakable seal 407, and bottom 412. The specimen treatment chamber is defined by the interior sidewall 410, the breakable seal 407, and an upper opening 413. The two chambers are connected to each other at the neck area 414. The cover means 408 comprises a handle section 415 and a threaded insert section 416 sized to fit the upper open end 413. A through hole 417 in the middle of the cover means holds the seal breaking stick 409. The seal breaking stick 409 and the through hole are sized to fit each other and when pushed from the upper end 418 the stick is capable of sliding downward and breaking the breakable seal 407 with the lower end 419 of the stick. The lower end 419 of the stick is shaped so that it is capable of breaking the seal. In this drawing, the lower end 419 of the stick is depicted as having a narrow, for example, a pointed edge, but those of skill in the art understand that any shape capable of breaking the seal is appropriate. The absorbent reagent member 404 comprises an upper end 420, a lower end 421, a test section 422 proximal to the upper end 420 comprising a reagent 423, and a wick section 424 proximal to the lower end 421.

When the cover means is removed from the device, a specimen is capable of being introduced into the specimen treatment chamber and mixed with the treatment buffer 406 to form a sample solution. When the cover means 408 and the seal breaking stick 409 are fit to the device, by pushing the upper end 418 of the stick 409, the stick is capable of sliding toward the lower end of the device and the lower end 419 of the seal breaking stick is capable of breaking the breakable seal 407. As a result, the sample solution flows from the specimen treatment chamber 405 to the test chamber 403 and contact the wick section of the absorbent reagent member 404. The sample solution flows from the wick section to the test section by capillary action and reacts with the test reagent 423 of the test section 422 and results in a test result of the presence or quantity of the test substance of the specimen.

FIG. 5 depicts another embodiment of a device of the invention. The device 500 is a modified version of the device 400 as depicted in FIG. 4. In addition to comprising a breakable seal 501 that separates the specimen treatment chamber 502 and the test chamber 503, a second breakable seal 504 retains the treatment buffer 505 inside the treatment buffer chamber 506 defined by the interior sidewall 507, breakable seal 501 and breakable seal 504. The seal breaking stick 508 is disposed above the breakable seal 504. By pressing the upper end 509 of the seal breaking stick, the stick is capable of sliding downward and breaking the breakable seals 504 and 501 consecutively. Breaking seal 504 enables mixing of a specimen introduced into the specimen treatment chamber 502 with the treatment buffer 505, and breaking seal 501 enables the sample solution flow from the specimen treatment chamber to the test chamber. At the test chamber 503, the sample solution contacts the wick section 511 of the absorbent reagent member 512, wicks up the absorbent reagent member to the test section 513 and reacts with the test reagent 514. As a result, a test result of the presence or quantity of the test substance of the specimen is produced.

Prior to being broken, the second breakable seal 504 separates the specimen treatment chamber 502 into two chambers, the treatment buffer chamber 506 proximal to the lower end and chamber 515 defined by the sidewall 507, the second breakable seal 504, and the upper opening 517. The chamber 515 may contain an additional treatment buffer for treating the specimen. For example, a test substance extraction buffer may be disposed in the chamber 515 to treat a specimen to extract the test substance from insoluble specimen components and a neutralizing solution may be stored in the treatment buffer chamber 506 to neutralize the extraction buffer.

The device of the invention may comprise one or more than one absorbent reagent members for testing one or simultaneously testing more substances in a specimen. For example, a device for fecal occult blood test may comprise a reagent member for detecting hemoglobin and another reagent member for detecting hapto-hemoglobin complex. Further, the reagent member may be in liquid, or dry form. In one embodiment of the invention, the reagent member of the test device is a liquid solution comprising reagents capable of reacting with analytes of the sample solution to be tested and producing an assay signal indicative of the presence or quantity of at least one analyte of the sample solution. In another embodiment of the invention, the assay reagent is a dry reagent comprising reagents capable of reacting with analytes of the sample solution to be tested and producing an assay signal indicative of the presence or quantity of at least one analyte of the sample solution. Dry reagents, air-dried or lyophilized, generally have a longer shelf life than liquid reagents. A preferred dry form of assay reagent is, for example, but not limited to, a dry reagent pad, a porous matrix containing the dry assay reagent. Such dry reagents are used for a variety of testing products, such as, for example, urine glucose, pH, creatinine, and alcohol. An exemplary example of a preferred dry reagent member is a lateral flow test strip.

The present invention also provides kits for detecting test substances in solid, semi-solid, or liquid specimens. For example, provided are kits that comprise a device of the present invention. The kits may also comprise more than one device of the present invention. The kits may further comprise treatment buffer. The treatment buffer may be contained in the specimen treatment module, or may be supplied separately in the appropriate container, such as, for example, a vial. The kits may further comprise instructions for testing for the presence of a substance in a specimen, and may further comprise instructions for obtaining specimen samples. The kits may further comprise reference samples that may be used to compare test results with the specimen samples. In certain exemplary examples, the kits comprise one or more specimen modules. These patient kits may further comprise instructions for obtaining specimen samples. The patient kits may comprise more than one specimen module in order for the patient to collect multiple samples, at either the same, or, for example, at different times. In one such example, the patient may collect a first sample on one day, and a second sample on another day. The patient kits may further comprise packaging for shipping or otherwise providing the sample in the specimen test module to the health care provider or laboratory.

### EXAMPLE 1

### Fecal Occult Blood Test

The presence of hemoglobin in feces can be indicative of gastrointestinal tract disorders associated with bleeding such as, for example, colorectal carcinoma, colon polyps, Crohn's disease, and ulcerative colitis. The present example provides a specimen treatment and test device, for example, the device as shown in FIGS. 1-3, that does not require liquid pipetting or transfer of the sample or sample solution.

The test reagent member of the example device is a lateral flow test strip based on immunochromatographic assay principle described in, for example, Rosenstein, et al. in U.S. Pat. No. 4,855,240. In general, a lateral flow test strip comprises a porous strip consisting from its upstream end to its downstream end a sample wick section, a test section comprising an upstream conjugate area and a downstream test area. A sample solution contacting the sample wick section is capable of migrating by capillary action through the conjugate area to the test area. The conjugate area in the example device comprises a movable color particle labeled anti-hemoglobin antibody. The test area comprises an immobilized second antibody of hemoglobin. When a sample solution contacts the sample wick section, the sample solution will first migrate to the conjugate area, and dissolve the movable labeled antibody. If hemoglobin is present in the sample solution, the hemoglobin will react with the labeled antibody and form a labeled antibody-hemoglobin complex. The labeled antibody-hemoglobin complex will migrate with the sample solution to the test area, where hemoglobin is again captured by the immobilized hemoglobin antibody forming a color band of labeled antibody-hemoglobin-immobilized antibody sandwich. Therefore, the presence of a color band at the test area indicates the presence of hemoglobin in the sample solution or blood in the test specimen. On the other hand, the absence of the color band at the test area indicates absence of hemoglobin in the sample solution or blood in the specimen. An internal control means is usually comprised in the lateral flow test device; a control band in a control area of the test section adjacent to the test area is formed by antibody-antigen binding independent of the test substance. The control band always presents in the end of a valid test procedure.

A fecal sample is collected and prepared for testing using the sample stick attached to the cap of the device of the invention. The sample stick is inserted into a fecal specimen at, for example, several different sites. Excess sample may be removed from the stick, if necessary, by gentle wiping with an absorbent tissue. The sample stick is inserted into the specimen treatment module and the cap is tightened securely. The specimen treatment module may then be shaken vigorously to obtain a liquid suspension of the sample. The specimen treatment module is then provided to a laboratory or other medical testing provider, or the test may be conducted by the patient. The specimen treatment module is then inserted into the test module, where the breakable seal of the specimen treatment module is broken. In one example, the seal is broken by moving toward the lower end of the test module, which has a seal breaking structure. In another example, once the specimen treatment module is inserted into the test module, the sample stick is pressed down using a movable connection to the cap, and pushes through the seal.

The sample solution is then allowed to flow into the test module. In certain embodiments, the sample solution flows more easily after loosening or removing the cap. In other embodiments, the sample solution flows more easily by sliding a plunger, attached to the upper end of the specimen treatment module, down the tube. In other examples, where the sample stick comprises an o-ring gasket, the o-ring creates positive pressure in the sample treatment module, and once the bottom seal is broken, the sample solution flows freely into the test module.

The device is kept in an upright position for the appropriate amount of time for testing, for example, 5 minutes, after which time the results may be read. Those of ordinary skill in the art may determine the appropriate amount of time for accurate results. A negative test is indicated when, for example, one rose pink color band appears in the control zone, meaning that the fecal sample does not contain a detectable level of human hemoglobin. A positive test is indicated when two rose-pink color bands appear, one in the test zone and one in the control zone. A positive result indicates that the specimen contains human hemoglobin. An invalid test is indicated where, after five minutes, no bands appear, or a test band appears without a control band appearing.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Singular forms “a”, “an”, and “the” include plural reference unless the context clearly dictates otherwise. Thus, for example, reference to “a subset” includes a plurality of such subsets, reference to “a nucleic acid” includes one or more nucleic acids and equivalents thereof known to those skilled in the art, and so forth. The term “or” is not meant to be exclusive to one or the terms it designates. For example, as it is used in a phrase of the structure “A or B” may denote A alone, B alone, or both A and B.

Unless defined otherwise, all technical and scientific terms used herein have the same meanings as commonly understood by one of ordinary skill in the art to which this invention belongs. Although any methods and systems similar or equivalent to those described herein can be used in the practice or testing of the present invention, the methods, devices, and materials are now described. All publications mentioned herein are incorporated herein by reference for the purpose of describing and disclosing the processes, systems and methodologies that are reported in the publications which might be used in connection with the invention. Nothing herein is to be construed as an admission that the invention is not entitled to antedate such disclosure by virtue of prior invention.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions that have been employed are used as terms of description and not of limitation, equivalents of the features shown and described, or portions thereof, are not excluded, and it is recognized that various modifications are possible within the scope of the invention. Embodiments of the invention are set forth in the following claims.

What is claimed is:

1. A specimen test device comprising
a. a specimen treatment module comprising a tubular part having a sidewall, an upper open end, and a lower end closed with a breakable seal; and
b. a test module comprising a container comprising an upper open end, a lower end, a sidewall, and a reagent member attached to the interior side of the sidewall;
wherein the specimen treatment module may be inserted into the upper open end of the test module.

2. The test device of claim 1, further comprising a treatment buffer.

3. The test device of claim 1, further comprising a cap.

4. The test device of claim 3, further comprising a specimen sampling stick.

5. The test device of claim 2, wherein the treatment buffer is pre-stored in the specimen treatment module and the open end of the specimen treatment module is removably closed with a cap.

6. The test device of claim 4, wherein the specimen sampling stick is attached to the cap.

7. The specimen test device of claim 1, wherein the lower end of the test module further comprises an upward edge for breaking the breakable seal of the specimen treatment module.

8. The specimen test device of claim 6, wherein the sampling stick is movably connected to the cap such that said sampling stick may be lowered toward the lower end of the specimen treatment module and break the breakable seal.

9. The specimen test device of claim 1, wherein said specimen is solid, semi-solid, or liquid.

10. The specimen test device of claim 1, further comprising a filter attached to the sidewalls of the specimen treatment module, wherein said filter may filter a liquid sample added to the specimen treatment module.

11. The specimen test device of claim 1, comprising at least 2 reagent members.

12. The specimen test device of claim 1, comprising from 3-5 reagent members.

13. The specimen test device of claim 1, wherein said reagent member comprises a wick section and a test section comprising an assay reagent.

14. The specimen test device of claim 1, wherein said reagent member detects the presence of an analyte that indicates that presence of a substance selected from the group consisting of fecal occult blood components, hapto-hemoglobin complex, antibodies, bacteria, viruses, enzymes, proteins, drugs, substances of abuse, allergens, pesticides, and pollutants.

15. The specimen test device of claim 1, wherein said reagent member detects the presence of fecal occult blood.

16. The specimen test device of claim 1, wherein said specimen treatment module further comprises a sealing structure on the exterior of the tubular part, wherein when the specimen treatment module is inserted into the test module, the sealing structure fits to the sidewalls of the test module.

17. The specimen test device of claim 4, wherein the sampling stick further comprises a plunger structure, wherein said plunger fits within the interior of the sidewalls of the specimen treatment module.

18. A method for testing an analyte in a specimen, comprising
a. treating the specimen with a treatment buffer in the specimen treatment module of the device of claim 1;
b. inserting the specimen treatment module into the test module of claim 1;
c. breaking the breakable seal of the specimen treatment module;
d. allowing the specimen and specimen treatment buffer to flow through the seal into the test module; and
e. detecting the presence of the analyte by reading the results on the reagent member.

19. The method of claim 18, further comprising closing the specimen treatment module with the cap and shaking the specimen treatment module.

20. The method of claim 18, further comprising opening the cap to allow the specimen and specimen treatment buffer to flow through the seal.

21. The method of claim 18, wherein said specimen is fecal matter.

22. The method of claim 21, wherein said reagent member detects analytes in fecal occult blood.

23. A kit comprising the specimen treatment module of claim 1.

24. The kit of claim 23, further comprising treatment buffer and a cap.

25. The kit of claim 23, further comprising a cap and a specimen sampling stick.

26. The kit of claim 25, further comprising treatment buffer.

27. The kit of claim 23, further comprising instructions.

28. The kit of claim 23, further comprising packaging for providing the device for testing.

29. The kit of claim 23, comprising 2-5 specimen treatment modules.

30. The kit of claim 23, further comprising the test module of claim 1.

31. The kit of claim 28, comprising from 1-10 specimen treatment modules and from 1-10 test modules.
